(19) United States
(12) Patent Application Publication
(10) Pub. No.: US 2006/0100483 A1
(43) Pub. Date: May 11, 2006

(54) Integrated viewing assembly for an endoscope

(76) Inventors: Scott A. Sundet, Edina, MN (US); Mark F. Brown, Plymouth, MN (US); Craig L. Riedl, Long Lake, MN (US); Steven E. Lombardi, New Brighton, MN (US); Thomas C. Barthel, Becker, MN (US)

Correspondence Address: OLSON & HIERL, LTD., 20 NORTH WACKER DRIVE, 36TH FLOOR, CHICAGO, IL, 60606, US

(21) Appl. No.: 10/984,165

(22) Filed: Nov. 9, 2004

(51) Int. Cl. A61B 1/00; A61B 1/12

(52) U.S. Cl. 600/131; 600/120; 600/156; 600/182; 600/114

(57) ABSTRACT

An integrated viewing assembly for an endoscope having an eyepiece and a handle incorporating a light source. The viewing assembly includes a hub having distal and proximal ends, a middle portion and an outer surface defining a port. The distal end defines a receiver in fluid communication with the port and adapted for onnection to an endotracheal tube. The port is adapted for connection to a gas source. A stylus is constructed and arranged to be fixedly connected to the hub within the receiver. Viewing and lighting conduits are carried by the hub and stylus. A coupler is located on the proximal end of the hub about the viewing conduit and is adapted for connection to the eyepiece. A connector on the middle portion of the hub connects to the handle. The receiver is located a predetermined distance from the longitudinal axis of the handle to facilitate tactile contact with a flange on the endotracheal tube connected to the receiver by the practitioner holding the handle. Preferably, the coupler, connector and hub are formed in one piece.

### FIELD OF THE INVENTION

The present invention relates generally to hand-held viewing endoscopes for assisting in endotracheal intubation procedures.

### BACKGROUND OF THE INVENTION

Endotracheal intubation is a common medical procedure by which a flexible plastic endotracheal breathing tube is inserted into a patient's trachea for providing oxygen or anesthetic gases to the lungs. Usually, the endotracheal tube is introduced into the patient's trachea after the patient has been sedated or has become unconscious, with initial placement of the endotracheal breathing tube often performed under emergency conditions. Therefore, it is desirable to have an apparatus that facilitates the accurate placement, as quickly as possible, of the endotracheal tube within the trachea of a patient.

Various devices have been used to aid in the placement of endotracheal tubes within the trachea of a patient, including viewing endoscopes. Viewing endoscopes use a slender, elongated stylus having viewing and lighting conduits that cooperate with a hub, eyepiece and light source to permit viewing within the trachea during the placement of the endotracheal tube. The lighting conduit of the endoscope carries light from the light source, typically located in a handle secured to the hub at a proximal end of the stylus, to the distal end of the stylus to illuminate a viewing area. The viewing conduit carries the illuminated image from the distal end of the stylus to the eyepiece, again typically located on the hub at the proximal end of the stylus. The stylus may comprise an elongated tubular member or lumen made of malleable material that can be bent or shaped to guide the endotracheal tube into the trachea of a patient.

With the viewing eyepiece and the handle containing or communicating with a light source attached to the hub, the malleable stylus is inserted into the endotracheal tube and the stylus and the endotracheal tube are thereafter inserted into the trachea of a patient while the practitioner views the illuminated interior of the trachea through the eyepiece. The endotracheal tube must be inserted past the patient's teeth and tongue and further past the epiglottis and vocal cords into the trachea. After the endotracheal tube is advanced past the vocal cords and into the patient's trachea, the distal end of the tube should be approximately 2 to 4 centimeters (about 1 to 2 inches) in front of the bifurcation of the trachea in order to ventilate both of the patient's lungs equally. The stylus is then removed from the endotracheal tube, and the endotracheal tube is connected to a supply conduit which then supplies oxygen or another other gas to the lungs of the patient.

The location of the endotracheal tube in relation to the stylus is vital to the proper placement of the endotracheal tube in front of the bifurcation of the trachea of the patient. To ensure that the distal end of the endotracheal tube is properly placed within the trachea during the viewing endoscopic procedure, the distal ends of the endotracheal tube and stylus should be approximately co-terminus in relation to one another prior to their insertion into a patient. To maintain the co-terminus relationship between the distal ends of the endotracheal tube and stylus, adapter stops have been utilized to affix the proximal end of the endotracheal tube to the stylus of the endoscope.

Adapter stops of prior art endoscopes generally comprise a housing distinct from the hub of the endoscope that defines a bore adapted to accept an insertion of the stylus there-through. With the stylus inserted through the bore of the housing, the housing can be moved adjustably along the stylus's length. A locking device is typically associated with the housing to temporarily affix the housing to the stylus in a predetermined location. The housing of the adapter stop also defines an access opening configured for attachment to the proximal end of the endotracheal tube, with the access opening generally co-axial with the bore. The access opening is typically in fluid communication with an inlet for connection to an oxygen or anesthetic gas source.

The stylus of the endoscope is inserted through the bore of the adapter stop and through the endotracheal tube, with the proximal end of the endotracheal tube thereafter attached to the access opening of the adapter stop. A gas source is then attached to the inlet of the adapter stop to provide oxygen or another gas to the patient during intubation. The adapter stop and attached endotracheal tube are then adjustably moved along the length of the stylus until the distal ends of the stylus and tube are about co-terminus with one another. The locking mechanism of the adapter stop is then actuated to affix the proximal end of the endotracheal tube to the stylus, thereby maintaining the co-terminus relationship between the distal ends of the stylus and tube. After the co-terminus relationship is established between the distal ends of the stylus and tube, both are inserted into the trachea of the patient.

Several disadvantages, however, are associated with prior art endoscopes using distinct adapter stops. Because the adapter stop is distinct from the hub, it comprises a separate component of the endoscope that must be handled by practitioners during intubation procedures. During an emergency procedure where time is of the essence, practitioners often forego use of the adapter stop due to the precious time consumed in connecting it to the endotracheal tube and stylus. In absence of the adapter stop, practitioners must then hold the endotracheal tube in relation to the stylus to maintain the co-terminus relation of their distal ends. Such practices by medical practitioners may jeopardize the crucial relationship required between the stylus and endotracheal tube for the proper placement of the endotracheal tube within a patient. Such practices may also jeopardize the provision of a gas source to the patient during intubation procedures because the component having the gas inlet to the endotracheal tube has been eliminated.

Furthermore, because the adapter stop is a separate component of the endoscope, it may dropped or misplaced by practitioners during the harried intubation procedures often occurring in emergency care. Such misplacement may result in improper use of the viewing endoscope by practitioners while intubating patients, or it may result in a breach of the sterility of the system if the adapter stop is recovered after misplacement.

In addition to the disadvantages associated with adapter stops relating to emergency intubation procedures, disadvantages also exist relating to ease of use of the endoscope. Because the endotracheal tube is attached to the adapter stop while placing the tube within the patient, the tube must be separated from the adapter stop after proper placement of the tube within a patient and prior to withdrawing the stylus of the endoscope from the endotracheal tube. The removal of the endotracheal tube from the stop is often cumbersome when the adapter stop is located on the stylus in a location spaced from the hub. The attachment or adjustment of the gas source at the inlet of the adapter stop may be cumbersome as well if the stop and hub are displaced a considerable distance from one another.

The practitioner typically holds the endoscope by a handle secured to the hub and must remove one or more hands from the handle to detach the endotracheal tube from the adapter stop, or to attach or adjust the gas source at the adapter stop's inlet. In removing his or her hands from the handle of the endoscope during an intubation procedure, the practitioner may encounter difficulty gripping the endoscope and may possibly jeopardize the proper placement of the endotracheal tube within a patient.

With regard to the handle of the endoscope, it is noted that many endoscopes utilize a handle that also comprises the light source of the system. The handle, typically attached to the hub of the endoscope, may include a battery power source and a light bulb for cooperation with the lighting conduit of the system. Within the medical industry, standards have been developed in relation to the structure of such handles so that a given handle may be utilized with a variety of medical devices requiring a light source. For example, the International Organization for Standardization (ISO) has promulgated a standard, namely, ISO 7376, for anesthetic and respiratory equipment that specifies general requirements for laryngoscopes and critical dimensions for the handle and lamp of hook-on type laryngoscopes. This standard is widely accepted and used within the medical industry.

A need therefore exists in the art for an endoscope that eliminates an adapter stop that is separate and distinct from the hub of an endoscope, thereby eliminating both the possibility of misplacement of the component during emergency intubation procedures and cumbersome procedures relating to the endotracheal tube or gas source connected to the adapter stop. A need also exists in the art for an endoscope that utilizes a standardized handle incorporating a light source and a standardized connection to the hub, and which is constructed and arranged so that the practitioner can disengage the adapter stop from the hub by manipulation from the hand holding the handle to release the endotracheal tube from the stylus and permit withdrawal of the stylus to leave the endotracheal tube in place in the patient. The present invention meets these needs.

### SUMMARY OF THE INVENTION

Numerous other advantages and features of the present invention will become readily apparent to those skilled in the art from the following detailed description of the preferred embodiment of the invention, the drawings, and the appended claims.

### BRIEF DESCRIPTION OF THE DRAWINGS

FIG. 1 shows a viewing endoscope embodying the present invention;

FIG. 2 is an exploded perspective view of the viewing endoscope of FIG. 1;

FIG. 3 is a front perspective view of the viewing endoscope of FIG. 1;

FIG. 4 is a longitudinal cross-sectional view of the hub of the viewing endoscope; and

FIG. 5 is a rear view of the hub of the viewing endoscope of FIG. 4.

### DETAILED DESCRIPTION OF THE PREFERRED EMBODIMENTS

While this invention can be embodied in many different forms, there are shown in the drawings and described in detail, preferred embodiments of the invention. The present disclosure is an exemplification of the principals of the invention and is not intended to limit the invention to the embodiments illustrated.

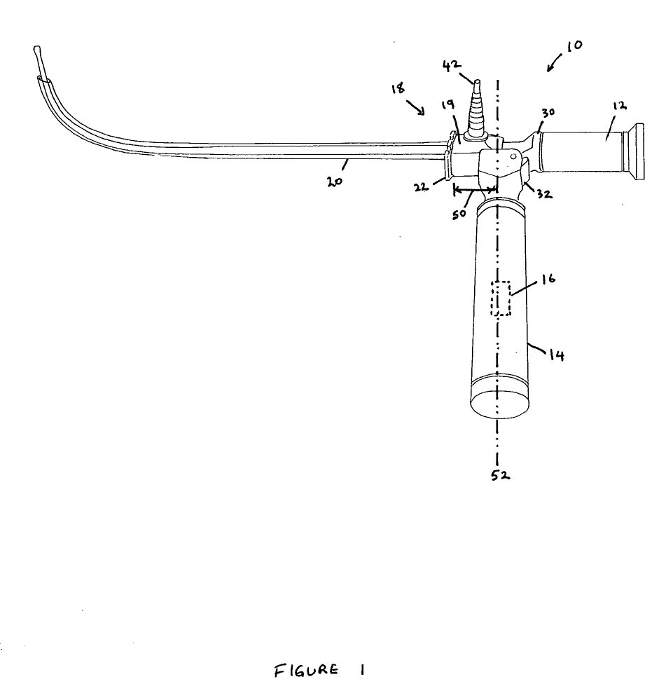

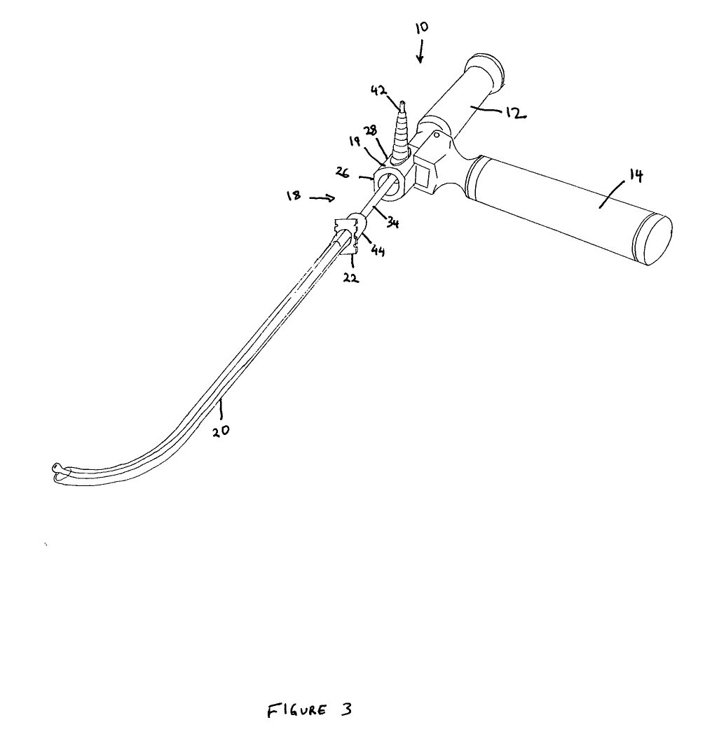

Referring to FIGS. 1, 2, and 3 of the drawings, there is shown a viewing endoscope 10 including an eyepiece 12, a handle 14 including a conventional light source 16 comprising a battery or batteries and a bulb, and a viewing assembly 18 operatively connected to a hub 19. The viewing endoscope 10 is associated with a generally conventional endotracheal tube 20 having at least one flange 22 on the adapter stop 44 on the end thereof proximate the hub 19.

The viewing endoscope 10 facilitates the viewing by a medical practitioner of the interior of a patient's trachea during an endotracheal intubation procedure. A viewing of the interior or the patient's trachea during an intubation procedure allows the medical practitioner to properly locate a distal end of the endotracheal tube therein.

The hub 19 has a proximal end 24 and a distal end 26 and a mesial or middle portion 28. In a preferred embodiment of the invention, the hub 19 is made of corrosion-resistant metal, such a aluminum, stainless steel, or an inert plastic. Because of the material composition of the hub 19, it is receptive to sterilization procedures that may include elevated temperature levels.

A coupler 30 is located on the proximal end 24 of the hub 19 and is constructed and arranged to receive the eyepiece 12. A connector 32 is located on the mesial or middle portion of the hub 19 and is provided with hook portions 35 that define a slot 33 in the connector 32. The connector 32 is constructed and arranged to cooperate with a conventional hook-on fitting on the handle 14 so as to secure the hub 19 to the handle 14. Preferably, the hub 19, coupler 30 and connector 32 are integrally formed.

Stylus 34 is secured at one end within receiver 40 defined by a bore in the distal end 26 of the hub 19 in a suitable manner, for example, by a threaded connection or by compression fit. The stylus 34 is a flexible elongated member made from a suitable material, for example, inert rubber or a polymeric material. The stylus 34 is flexible so that it can be bent in various directions without retaining a memory. Such flexibility is desirable during intubation procedures to enable the practitioner to insert the stylus 34 through the mouth of the patient and into the trachea.

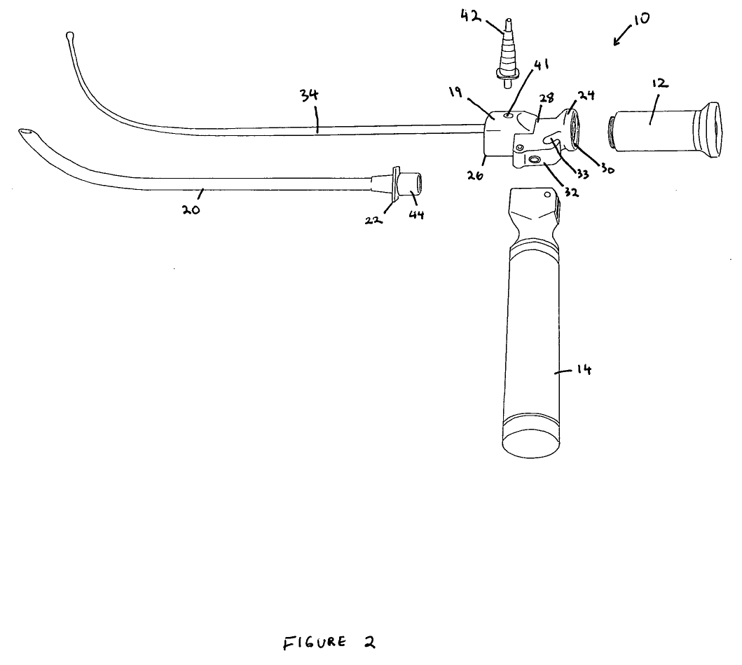

The connection of the eyepiece 12 to the hub 19 is facilitated by the coupler 30 (best shown in FIGS. 2, 4, and 5), which comprises a threaded receiver cooperating with complementary threads on the end of the eyepiece 12. The complementary threads for engaging the eyepiece 12 to the coupler 30 on the hub 19 may be internal in the coupler 30 and external on the eyepiece, or vice versa. It will be apparent to persons skilled in the art that other forms of engagement components can be employed to secure the eyepiece 12 to the hub 19, for example, a compression fit or bayonet connection.

A port 41 (best shown in FIG. 2) is provided in the hub 19 to provide fluid communication from the outer surface of the hub 19 to the receiver 40. The port 41 receives a fitting 42, which is adapted to be connected to a gas source (not shown). The bore, which is in fluid communication with the port 41, of the receiver 40 circumscribes the stylus 34 and is constructed and arranged to accept insertion of the adapter stop 44 on the endotracheal tube 20 therein.

The endotracheal tube 20 includes a portion of substantially uniform cross section and has the adapter stop 44 at one end which is constructed and arranged to fit within the receiver 40 of the hub 19. Basically in use, the endotracheal tube 20 and the adapter stop 44 secured on the end thereof are slipped over the stylus 34 until the end of the stop 44 is received in the receiver 40. The engaged position of the adapter stop 44 in the hub 19 is a predetermined distance 50 (FIG. 1) from the longitudinal axis 52 of the handle to facilitate tactile contact by the user with the flange or flanges 22 on the adapter stop 44 for disengaging the endotracheal tube 20 from the stylus 34 when desired during an intubation procedure. The practitioner can utilize a finger on the hand engaging the handle 14 to exert an outward force on the flange or flanges 22 without removing his/her hand from the handle 14.

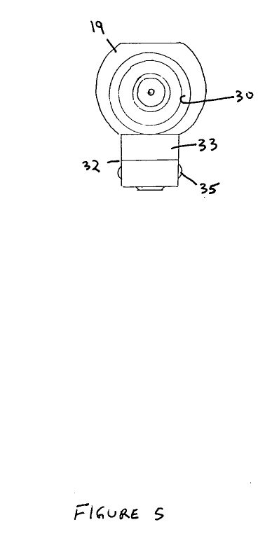

As better shown in FIGS. 2, 4, and 5, the connector 32 has a slot 33 which engages with the hook-on fitting on the handle 14 to secure the hub 19 and the handle 14 to one another. The slot 33 may be formed by separate hook portions 35, as shown in FIG. 5, or by a single hook portion extending the width of the connector 32.

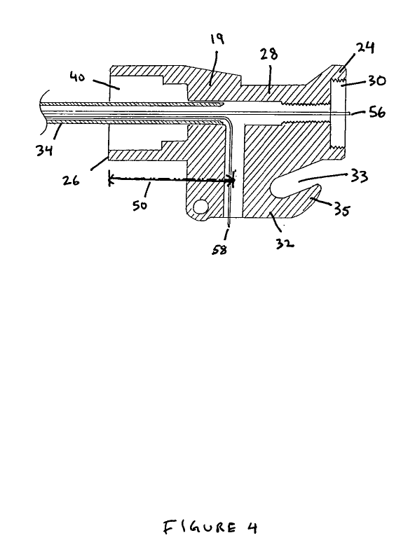

With reference to FIG. 4, the endoscope 10 includes a viewing conduit 56 and a lighting conduit 58 within the hub 19 that cooperate, respectively, with the eyepiece 12 and the light source 16 in the handle 14. The viewing conduit 56 extends between the eyepiece 12 and the distal end of the stylus 34. The lighting conduit 58 extends from the light source 16 in the handle 14, through a passage in the middle portion of the hub 19 to the distal end of the stylus 34. The lighting conduit 58 of the endoscope 10 carries light from the light source 16 in the handle 14 to the distal end of the stylus 34 to illuminate a viewing area within the trachea while the viewing conduit 56 carries the illuminated image from the distal end of the stylus to the eyepiece 12 for observation by a medical practitioner. Thus, the endoscope 10 facilitates viewing by the medical practitioner of the interior of the trachea of a patient during an endotracheal intubation procedure. A viewing of the interior of the trachea of the patient during an intubation procedure allows the medical practitioner to properly locate a distal end of the endotracheal tube therein.

In use, the medical practitioner will place the endotracheal tube 20 on the stylus 34, with the stop 44 inserted into the receiver 40 in the hub 19. A gas source will be connected to the fitting 42 that has been inserted into the port 41. The stylus 34 and the endotracheal tube 20 are advanced through the mouth into the trachea of the patient. After proper positioning of the endotracheal tube 20 in the patient, the medical practitioner will disconnect the endotracheal tube 20 from the receiver 40 of the hub 19 by tactile contact, i.e., by pushing outwardly on the flange or flanges 22 on the adapter stop 44 by a finger on the hand holding the handle 14 of the viewing endoscope 10. The medical practitioner can then withdraw the stylus 34 from the endotracheal tube 20, leaving the endotracheal tube 20 properly positioned in the trachea of the patient.

While we have shown a presently preferred embodiment of the present invention, it will be apparent to persons skilled in the art that the invention may be otherwise embodied within the scope of the following claims.

What is claimed is:

1. A viewing endoscope having an eyepiece and a handle incorporating a light source,
a hub having a distal end and a proximal end, a middle portion and an outer surface defining a port, the distal end defining a receiver in fluid communication with the port and adapted for connection to an endotracheal tube, the port adapted to be connected to a gas source;
a stylus having a proximal end constructed and arranged to be fixedly connected to the hub within the receiver;
viewing and lighting conduits carried by the hub and stylus, the viewing conduit extending from the proximal end of the hub to a distal end of the stylus and the lighting conduit communicating with the light source and extending from the middle portion of the hub to the distal end of the stylus;
a coupler located on the proximal end of the hub about the viewing conduit and adapted for connection to the eyepiece; and
a connector located on the middle portion of the hub about the lighting conduit and adapted for connection to the handle.

2. The endoscope of claim 1 wherein the receiver is located at a predetermined distance from the longitudinal axis of the handle to facilitate tactile contact with the endotracheal tube connected to the receiver by a user holding the handle of the endoscope.

3. The endoscope of claim 2 wherein the tactile contact with the endotracheal tube comprises an outward force applied to at least one flange located on the endotracheal tube.

4. The endoscope of claim 1 wherein the receiver comprises a distal end surface defining a bore in fluid communication with the port, the bore circumscribing the stylus and constructed and arranged to accept the endotracheal tube therein.

5. The endoscope of claim 1 wherein the hub, coupler and connector are integrally formed.

6. The endoscope of claim 1 wherein the hub is comprised of a corrosion-resistant metal.

7. The endoscope of claim 1 wherein the hub is comprised of an inert plastic.

8. The endoscope of claim 1 wherein the coupler is provided with a threaded surface for threaded connection to the viewing eyepiece.

9. The endoscope of claim 1 wherein the connector is constructed and arranged for engagement with a standard hook-on fitting on the handle.

10. In an endoscope having an eyepiece and a standardized handle incorporating a light source,
a unitary hub having distal and proximal ends, a mesial portion and an outer surface defining a port, the distal end having an end surface defining a bore in fluid communication with the port, the bore constructed and arranged to accept insertion of an endotracheal tube therein and the port adapted for connection to a gas source;
a stylus having a proximal end fixably connected to the hub's distal end and circumscribed by the bore of the hub;
viewing and lighting conduits carried by the hub and stylus, the viewing conduit extending from the proximal end of the hub to a distal end of the stylus and the lighting conduit communicating with the light source and extending from the middle portion of the hub to the distal end of the stylus;
a coupler located on the hub's proximal end about the viewing conduit and adapted for connection to the eyepiece; and
a connector located on the middle portion of the hub about the lighting conduit and adapted for connection to the standardized handle incorporating a light source.

11. The endoscope of claim 10 wherein the distal end surface of the hub is located at a predetermined distance from the longitudinal an axis of the handle to facilitate tactile contact with the endotracheal tube inserted within the bore by a user holding the handle connected to the endoscope.

12. The endoscope of claim 11 wherein the tactile contact with the endotracheal tube includes an outward force applied to at least one flange located on the endotracheal tube.

13. The endoscope of claim 11, wherein an adapter stop is secured on the end of the endotracheal tube and at least one flange projects from the adapter stop.

14. The endoscope of claim 10 wherein the hub is comprised of a corrosion-resistant metal.

15. The endoscope of claim 11 wherein the hub is comprised of an inert plastic.

16. The endoscope of claim 10 wherein the coupler is provided with a threaded surface for threaded connection to the viewing eyepiece.

17. The endoscope of claim 10 wherein the connector is constructed and arranged for engagement with a standard hook-on fitting on the handle.

18. A method of placing an endotracheal tube in a patient comprising the steps of:
providing a viewing endoscope having a hub with an outer surface defining a port and a a receiver circumscribing a stylus, the port in fluid communication with the receiver and adapted for connection to a gas source, the receiver adapted for connection to the endotracheal tube and the hub having an eyepiece and a handle including a light source;
inserting the stylus into the endotracheal tube and connecting the endotracheal tube with the receiver of the hub;
connecting a gas source to the port of the hub;
advancing the stylus and endotracheal tube into the patient;
disconnecting the endotracheal tube from the receiver of the hub through tactile contact by a hand holding the handle of the endoscope; and
withdrawing the stylus from the endotracheal tube and patient.

19. The method of claim 18 wherein the tactile contact comprises applying an outward force against at least one flange located on the endotracheal tube proximate the handle.
